(19) United States
(12) Patent Application Publication
(10) Pub. No.: US 2025/0387250 A1
(43) Pub. Date: Dec. 25, 2025

(54) IMPLANTABLE MEDICAL CLOSURE DEVICE AND FLEXIBLE DEPLOYMENT DEVICE FOR DEPLOYING THE SAME INTO VASCULATURE

(71) Applicant: W. L. Gore & Associates, Inc., Newark, DE (US)

(72) Inventors: Edward H. Cully, Clarksville, VA (US); Cody L. Bliss, Peoria, AZ (US); Jeffrey B. Duncan, Flagstaff, AZ (US)

(21) Appl. No.: 19/243,124

(22) Filed: Jun. 19, 2025

Related U.S. Application Data
(60) Provisional application No. 63/661,790, filed on Jun. 19, 2024.
(60) Provisional application No. 63/661,787, filed on Jun. 19, 2024.
(60) Provisional application No. 63/825,863, filed on Jun. 18, 2025.

(51) Int. Cl. A61F 2/966; A61B 17/00; A61F 2/95

(52) U.S. Cl. CPC A61F 2/9662; A61B 17/0057; A61F 2/9517; A61B 2017/00004; A61B 2017/00575; A61B 2017/00623; A61B 2017/0065; A61F 2002/9511; A61F 2250/0098

(57) ABSTRACT

An implantable medical device for closure of a tissue tract/opening in a vessel wall includes a constraint disposed around and constraining the implantable medical device in a constrained configuration and one or more deployment lines attached to the constraint. A flexible hollow body is coupled proximate a midpoint of the implantable medical device. The implantable medical device and flexible hollow body are operable to transition (pivot) from a parallel, side-by-side arrangement to be substantially angled relative to one another. Retracting the one or more deployment lines causes the constraint to release the implantable medical device to transition from the constrained configuration to an expanded configuration.

### CROSS REFERENCE TO RELATED APPLICATION

The present application claims the benefit of U.S. Provisional Application No. 63/661,790, filed Jun. 19, 2024, U.S. Provisional Application No. 63/661,787, filed Jun. 19, 2024, and U.S. Provisional Application No. 63/825,863, filed Jun. 18, 2025, which are incorporated herein by reference in their entireties for all purposes.

### FIELD

The present disclosure relates generally to apparatuses, systems, and methods relating to medical device deployment. Specifically, the disclosure relates to apparatuses, systems, and methods relating to deploying an implantable medical device in a body, such as in a blood vessel.

### BACKGROUND

Vascular disease may occur at any location in a vessel. Stents are examples of expandable endoluminal prosthetic devices, which may be used to treat certain diseases or conditions by maintaining, opening, or dilating the vessel at such locations. However, placement and deployment of such prosthetic devices can often be difficult or problematic, especially when there are multiple devices that need to be deployed or when many steps are involved in the deployment process. As such, there is a need for deployment devices that are flexible and adjustable to improve the accuracy of the deployment of the stents and other implantable medical devices.

### SUMMARY

Deployment devices are provided to have a hollow body through which deployment lines are extended, and the deployment lines are coupled with an implant that is constrained in one or more constraint(s). The deployment devices as discussed herein may be used in various applications, including but not limited to surgical applications. For example, the deployment devices may be used in conjunction with introducers, guidewires, and/or any other suitable devices for introducing or implanting medical devices and apparatuses into and out of the body during surgical procedures.

According to a first aspect (“Aspect 1”), an implantable medical device system includes an implantable medical device having a midpoint along a length of the implantable medical device, at least one constraint disposed around and constraining the implantable medical device in a constrained configuration, a deployment device including a flexible hollow or tubular body, and one or more deployment lines attached to the at least one constraint, wherein at least one of the one or more deployment lines extend along the flexible hollow body and past a distal end of the flexible hollow body to operatively connect with the at least one constraint along the midpoint of the implantable medical device. The implantable medical device and flexible hollow body are operable to transition from a substantially parallel (e.g., side-by-side) orientation with the implantable device extending next to the flexible hollow or tubular body to an angled orientation (e.g., orthogonal) relative to one another. And, retracting the one or more deployment lines is operable to cause the at least one constraint to release the implantable medical device to transition from the constrained configuration to an expanded configuration with a greater cross-sectional area than the constrained configuration.

According to another aspect (“Aspect 2”), further to Aspect 1, the at least one constraint includes a first constraint attached to a first deployment line of the one or more deployment lines and a second constraint attached to a second deployment line of the one or more deployment lines.

According to another aspect (“Aspect 3”), further to Aspect 2, the implantable medical device transitions from the constrained configuration to the expanded configuration by first expanding an intermediate portion of the implantable medical device located between a first end portion and a second end portion of the implantable medical device when the first deployment line and the second deployment line are partially retracted, before expanding the first end portion and the second end portion of the implantable medical device when the first deployment line and the second deployment line are fully retracted.

According to another aspect (“Aspect 4”), further to Aspect 1, the at least one constraint is a single constraint, and a first deployment line of the one or more deployment lines and a second deployment line of the one or more deployment lines are attached to two opposing ends of the single constraint.

According to another aspect (“Aspect 5”), further to Aspect 4, the implantable medical device transitions from the constrained configuration to the expanded configuration by first expanding a first end portion and a second end portion of the implantable medical device when the one or more deployment lines are partially retracted, before expanding an intermediate portion of the implantable medical device located between the first end portion and the second end portion when the one or more deployment lines are fully retracted.

According to another aspect (“Aspect 6”), further to Aspect 1, the implantable medical device has a first end portion and a second end portion, and the implantable medical device includes a first radiopaque marker disposed at or near the first end portion and a second radiopaque marker disposed at or near the second end portion.

According to another aspect (“Aspect 7”), further to any preceding Aspect, the implantable medical device comprises a self-expanding stent-graft.

According to another aspect (“Aspect 8”), further to Aspect 7, the stent-graft includes a graft component comprising an outer layer and an inner layer, the outer layer includes at least one outer aperture, the inner layer includes at least one inner aperture, and the outer aperture and the inner aperture are longitudinally misaligned with respect to each other.

According to another aspect (“Aspect 9”), further to Aspect 8, the deployment device includes a guidewire that is insertable into an internal channel of the implantable medical device through the outer layer and the inner layer.

According to another aspect (“Aspect 10”), further to any preceding Aspect, the flexible hollow body has a first end and a second end, and the implantable medical device is pivotably disposed adjacent to the second end of flexible hollow body.

According to another aspect (“Aspect 11”), further to Aspect 10, the one or more deployment lines are attached to a handle disposed adjacent to the first end of the flexible hollow body, wherein the handle has a greater width than the flexible hollow body.

According to another aspect (“Aspect 12”), further to Aspect 11, the handle is a pull ring.

According to another aspect (“Aspect 13”), further to any preceding Aspect the deployment device further comprises an implantable closure device disposed between the flexible hollow body and the implantable medical device, the implantable closure device comprising a body made of a bioabsorbable material and having an opening that is distensible in one or more directions, wherein the body self-transitions from a first configuration to a second configuration in the absence of a force applied to open the opening, wherein the opening is substantially open in the first configuration and substantially closed in the second configuration.

According to another aspect (“Aspect 14”), further to Aspect 13, the implantable closure device further includes a biocompatible adhesive applied to a surface of the body.

According to another aspect (“Aspect 15”), further to Aspect 9, the implantable medical device further comprises a removable guidewire tube through which the guidewire is disposed, wherein the removable guidewire tube is withdrawn from the implantable medical device or the flexible hollow body prior to the implantable medical device or the flexible hollow body being advanced to a treatment site.

According to another aspect (“Aspect 16”), a method for delivering and deploying an implantable medical device system into vasculature of a patient to close an opening formed in a vessel wall of the vasculature includes extending a guidewire percutaneously into the vasculature through the opening formed in the vessel wall. An implantable medical device is delivered through the opening into the vasculature along the guidewire using a deployment device coupled to a midpoint of the implantable medical device, wherein the guidewire is received through a flexible hollow body of the deployment device and through a portion of the implantable medical device, the implantable medical device initially being substantially parallel to the flexible hollow body and then tracking over the guidewire to longitudinally align with the vasculature during delivery into the vasculature after the implantable medical device passes through the opening. The implantable medical device is centered on the opening formed in the vessel wall. The implantable medical device is deployed from a constrained configuration to an expanded configuration to occlude the opening in the vessel wall and while permitting blood flow through a lumen of the implantable medical device.

According to another aspect (“Aspect 17”), further to Aspect 16, deploying the implantable medical device causes substantial occlusion of a tissue tract proximal the opening in the vessel wall.

According to another aspect (“Aspect 18”), further to Aspect 16 or 17, the deployment device includes at least one constraint maintaining a first portion of the implantable medical in a constrained configuration prior to deployment of the implantable medical device, the at least one constraint being attached to one or more deployment lines, and further wherein deploying the implantable medical device includes tensioning the one or more deployment lines.

According to another aspect (“Aspect 19”), further to Aspect 18, deploying the implantable medical device causes the implantable medical device to transition from the constrained configuration to the expanded configuration by first expanding an intermediate portion of the implantable medical device located between a first end portion and a second end portion of the implantable medical device when the one or more deployment lines are partially retracted, before expanding the first end portion and the second end portion of the implantable medical device when the one or more deployment lines are fully retracted.

According to another aspect (“Aspect 20”), further to Aspects 18 or 19, the at least one constraint is a single constraint, and a first deployment line of the one or more deployment lines and a second deployment line of the one or more deployment lines are attached to two opposing ends of the single constraint.

According to another aspect (“Aspect 21”), further to Aspect 20, deploying the implantable medical device causes the implantable medical device to transition from the constrained configuration to the expanded configuration by first expanding a first end portion and a second end portion of the implantable medical device when the first deployment line and the second deployment line are partially retracted, before expanding an intermediate portion of the implantable medical device located between the first end portion and the second end portion when the first deployment line and the second deployment line are fully retracted.

According to another aspect (“Aspect 22”), further to any one of Aspects 16 to 21, the implantable medical device further includes a stent graft that is transitionable from the constrained configuration to the expanded configuration and a body made of a bioabsorbable material having an opening that is distensible in one or more directions, the body being configured to self-transition from a first configuration to a second configuration during deployment of the implantable medical device, wherein the opening of the body is substantially open in the first configuration and substantially closed in the second configuration, and further wherein following deployment of the stent graft the body made of bioabsorbable material is positioned in the tissue tract and/or between the stent graft and the vessel wall.

The foregoing examples are just that, and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 shows a cross-sectional view of a portion of a deployment device implemented with a stent-graft in a constrained configuration according to embodiments disclosed herein;

FIG. 2A shows a side view of an implantable medical device such as a stent-graft in a constrained configuration according to embodiments disclosed herein;

FIG. 2B shows a side view of an implantable medical device such as a stent-graft in an expanded configuration according to embodiments disclosed herein;

FIG. 2C shows a cross-sectional view of a portion of an implantable medical device such as a stent-graft receiving a guidewire through the graft component according to embodiments disclosed herein;

FIG. 2D shows a cross-sectional view of a portion of an implantable medical device such as a stent-graft receiving a guidewire via a removable guidewire tube through the graft component according to embodiments disclosed herein;

FIG. 3A shows a side view of an implantable medical device when transitioning from the constrained configuration to the expanded configuration in a middle-first expansion pattern according to embodiments disclosed herein;

FIG. 3B shows a side view of an implantable medical device when transitioning from the constrained configuration to the expanded configuration in an edge-first expansion pattern according to embodiments disclosed herein;

FIG. 4A shows a side view of a deployment device according to embodiments disclosed herein;

FIG. 4B shows a side view of the deployment device when the implantable medical device is pivoted to be substantially parallel to the flexible hollow body according to embodiments disclosed herein;

FIGS. 5A through 5I show cross-sectional view of a portion of the tissue region surrounding a vasculature during a procedure performed using the deployment device and the implantable medical device according to embodiments disclosed herein;

FIG. 6 shows a side view of a deployment device for delivery of an implantable device according to embodiments disclosed herein;

FIG. 7 shows a cross-sectional view of a portion of the tissue region surrounding a vasculature after the implantable medical device and the implantable device are implanted according to embodiments disclosed herein;

FIG. 8A shows a top view of an implantable device in a substantially open configuration according to embodiments disclosed herein; and

FIG. 8B shows a top view of an implantable device in a substantially closed configuration according to embodiments disclosed herein.

### DETAILED DESCRIPTION

### Definitions and Terminology

This disclosure is not meant to be read in a restrictive manner. For example, the terminology used in the application should be read broadly in the context of the meaning those in the field would attribute such terminology.

With respect to terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example. In the event it is determined that individuals having ordinary skill in the relevant arts would not readily ascertain values for such reasonably small differences, the terms “about” and “approximately” can be understood to mean plus or minus 10% of the stated value.

### Description of Various Embodiments

Persons skilled in the art will readily appreciate that various aspects of the present disclosure can be realized by any number of methods and apparatuses configured to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

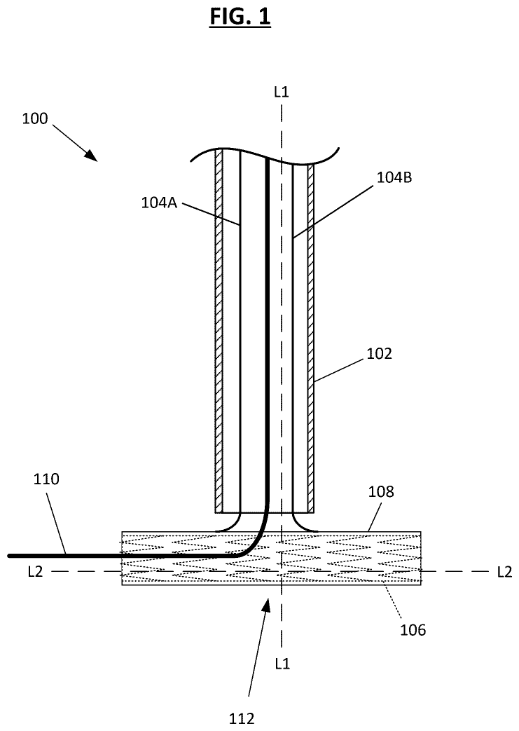

FIG. 1 shows an example of a deployment device 100 according to embodiments disclosed herein, provided as an example of the various features of the deployment device and, although the combination of those illustrated features is clearly within the scope of invention, that example and its illustration is not meant to suggest the inventive concepts provided herein are limited from fewer features, additional features, or alternative features to one or more of those features of the material shown in the figures and may include the other features such as being formed into three-dimensional structures such as shown in subsequent figures as disclosed herein. It should also be understood that the reverse is true as well. One or more of the components depicted in FIG. 1 can be employed in addition to, or as an alternative to components depicted in other figures as discussed herein.

Discussed herein are devices and methods for deploying an implantable medical device, such as a stent or stent-graft for closing an access site and access path for a vasculature such as a blood vessel, which is formed through the skin and the tissue region adjacent to the vasculature. The stent may be made of a nitinol (NiTi) material including but not limited to wires of nickel-titanium shape memory alloy(s) for medical devices and surgical implants in accordance with the ASTM F2063 standards, for example. Advantageously, the implantable devices and associated methods can be implemented to preserve flow through the vasculature, while also reliably and quickly closing off wounds, or openings, formed at an access site for accessing the vasculature. In various embodiments, the implantable device also provides a convenient re-access point, where the device permits one or more introducer(s), guidewire(s) and/or other endoluminal prostheses to be re-introduced through the device following implantation. Hereinafter, the implantable medical device may also be referred to as a closure device, a medical closure device, an implantable medical closure device, or a self-expanding medical device, for example.

Generally, as part of an endoluminal treatment method, the skin and the tissue region are penetrated or opened to form an access site or access path through the skin, muscle, fascia, organs, or other body tissue, to the vasculature. Referring to FIG. 1, the deployment device 100 includes a flexible hollow body 102, and an implantable medical device 106, such as a stent-graft, one or more deployment lines 104 (e.g., first and second lines 104A and 104B) and at least one constraint 108 (such as a sheath, fibers, tube, etc.) that constrains the implantable medical device 106 until the implantable medical device 106 is deployed. In some examples, the flexible hollow body 102 may be substantially tubular to define a flexible tubular body.

The constraint is optionally a knit sleeve configured to unravel, or deconstruct, by tensioning one or more deployment line(s). Examples of suitable knit sleeve constraints include those associated with the GORE® VIABAHN® Endoprosthesis (W. L. Gore & Associates, Inc,). The constraint may also be a releasable sheath design. Suitable examples include those associated with Conformable GORE® TAG® Thoracic Endoprosthesis (W. L. Gore & Associates, Inc.), although a variety of constraint designs are contemplated.

The deployment device 100 may further include a guidewire 110 that extends through the flexible hollow body 102 and the implantable medical device 106 that is to be deployed.

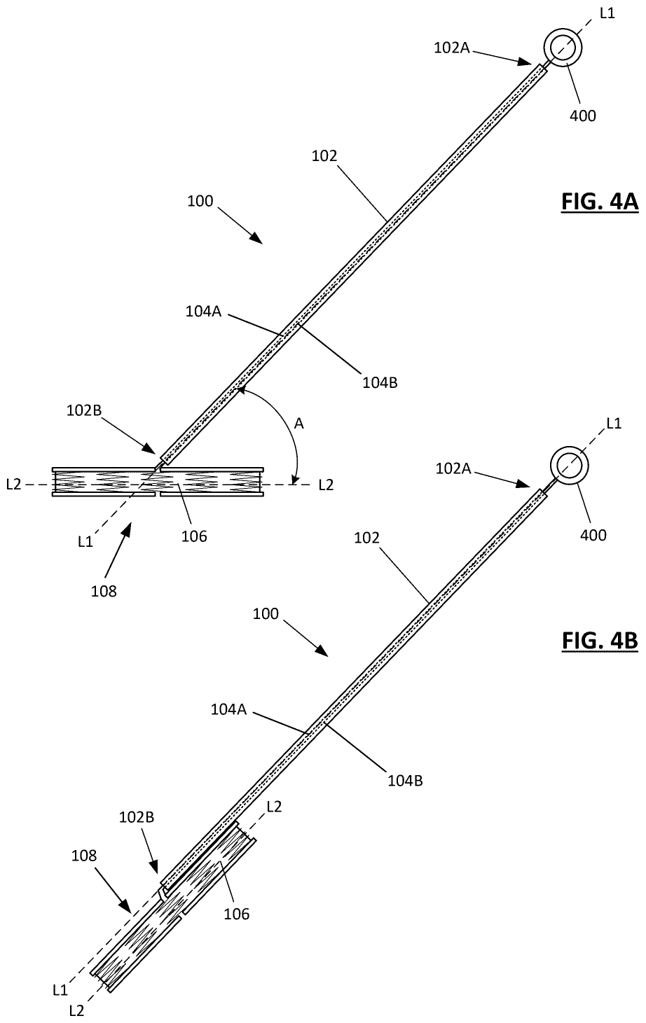

According to some examples, the flexible hollow body 102 may be a catheter, a tube, a conduit, or any substantially elongated member with a channel extending therethrough that is capable of receiving the guidewire 110 and the deployment line(s) 104. The flexible hollow body 102 generally exhibits sufficient column strength to push, or direct, the flexible hollow body 102 through the access site (e.g., through the introducer and along the guidewire) to the vasculature in need of treatment/sealing. At least one of the one or more deployment lines 104 extends along the flexible hollow body 102 and extends past a distal end (such as a second end 102B as shown in FIGS. 4A-B) of the flexible hollow body 102 and operatively connects with the at least one constraint 108 along a midpoint 112 of the implantable medical device 106.

FIG. 1 shows the deployment device 100 with a flexible hollow body 102, one or more deployment lines 104, an implantable medical device 106 that is to be deployed, and at least one constraint 108. The figure also shows a guidewire 110 that passes through the flexible hollow body 102 and a portion of the implantable medical device 106 (as well as a portion of the constraint 108) and is used for guiding the deployment device 100 during the deployment process of the implantable medical device 106, as further explained herein. The implantable medical device 106 is rotatably and/or pivotably disposed adjacent to the flexible hollow body 102. The constraint 108 is disposed around and constraining the implantable medical device 106 in a constrained configuration. The one or more deployment line(s) 104 (shown as two deployment lines 104A and 104B in the figure) extend through the flexible hollow body 102 and are attached to the constraint 108. The flexible hollow body 102 defines a longitudinal axis L1-L1, and the implantable medical device 106 defines another longitudinal axis L2-L2.

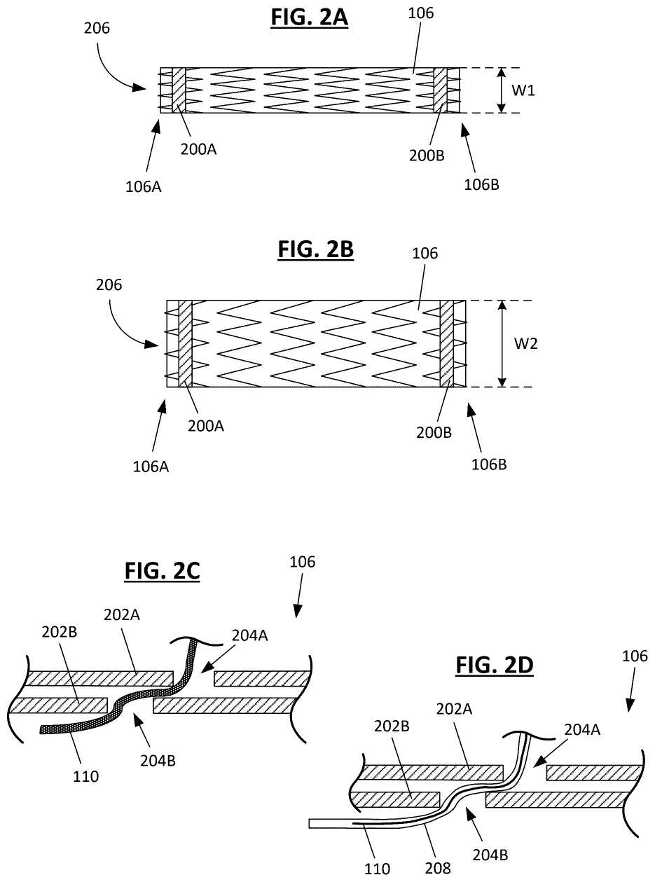

FIGS. 2A and 2B show the implantable medical device 106 in two different configurations. The implantable medical device 106 may be in a form similar to a stent or stent-graft having a stent component(s) and a graft component(s) attached together, or any other suitable self-expanding medical device that may be implemented for the desired access site-occlusion treatment. In FIG. 2A, the implantable medical device 106 is shown in a constrained configuration while being constrained by the constraint 108 to have a first cross-sectional area or width (W1). In FIG. 2B, the implantable medical device 106 is shown in an expanded configuration with a second cross-sectional area or width (W2) that is greater than that of the implantable medical device 106 in the constrained configuration. The length of the implantable medical device 106 may remain substantially the same in both configurations, as measured along the longitudinal axis L2-L2. As such, in the expanded configuration, an internal channel 206 of the implantable medical device 106 has a greater volume than in the constrained configuration.

In some examples, retracting the implantable medical device 106 along the guidewire 110 may cause the implantable medical device 106 to pivot with respect to the flexible hollow body 102, for example at the midpoint 112 of the implantable medical device 106. In some examples, the guidewire 110 may extend into the implantable medical device 106 at the midpoint 112 and protrude from the implantable medical device 106, such that the position of the implantable medical device 106 may substantially follow the direction of the guidewire 110. Also, in some examples, further or fully retracting the one or more deployment lines 104 may cause the constraint 108 to release the implantable medical device 106 to transition from the constrained configuration (smaller cross-sectional area) to an expanded configuration (greater cross-sectional area). In some examples, when the deployment lines 104 are pulled using a first amount of force that is sufficient to make the deployment lines 104 taut and also maintains the constraint 108 in the configuration that causes the implantable medical device 106 to be in the constrained configuration, the first amount of force causes the implantable medical device 106 to pivot with respect to the flexible hollow body 102. Subsequently, when the deployment lines 104 are pulled using a second amount of force that is greater than the first amount of force while the deployment lines 104 are taut, the second amount of force causes the implantable medical device 106 to be released from the constraint 108 as the constraint 108 is retracted in the direction in which the deployment lines 104 are pulled. In some examples, As the constraint 108 is retracted, the portion of the implantable medical device 106 that is not constrained by the constraint 108 is allowed to self-transition or self-expand as the implantable medical device 106 is being deployed. The implantable medical device 106 in the expanded configuration provides a seal or closure for the vasculature in which the implantable medical device 106 is deployed.

In some examples, the implantable medical device 106 has a first end portion 106A and a second end portion 106B. The implantable medical device 106 also includes a first radiopaque marker 200A disposed at or near the first end portion 106A and a second radiopaque marker 200B disposed at or near the second end portion 106B. The radiopaque markers 200 may be made of any suitable radiopaque material that can be detected using X-rays or similar radiation. Beneficially, the radiopaque markers 200 located at or near the two ends of the implantable medical device 106 allows for the medical device 106 to be detected even after it is implanted, such that the medical device 106, which may be a stent-graft, can be easily re-accessed using the guidewire 110 as needed.

FIG. 2C shows a portion of the implantable medical device 106, more specifically a graft component 202 of the self-expanding stent-graft that forms the implantable medical device 106, according to some examples. The graft component 202 has an outer layer 202A and an inner layer 202B, where the outer layer 202A includes at least one outer opening or aperture 204A extending through the layer 202A, and the inner layer 202B includes at least one inner opening or aperture 204B extending through the layer 202B. The outer aperture 204A and the inner aperture 204B are longitudinally misaligned with respect to each other. That is, the positions of the apertures 204A and 204B may be located at different points along the longitudinal axis L2-L2 of the implantable medical device 106.

In some examples, as shown in FIG. 2D, a removable guidewire tube 208 may be disposed around the guidewire 110 such that the guidewire 110 is received within or through the removable guidewire tube 208. The removable guidewire tube 208 may extend through the opening(s) or aperture(s) in the wall (such as the outer opening or aperture 204A extending through the layer 202A and the inner opening or aperture 204B extending through the layer 202B of the graft component 202) of the implantable medical device 106 and through the implantable medical device 106 for insertion of the guidewire 110 through a lumen or internal channel 206 of the implantable medical device 106. The removable guidewire tube 208 may be removed, retracted, or withdrawn from the implantable medical device 106 and/or the flexible hollow body 102 prior to the implantable medical device 106 and/or the flexible hollow body 102 being advanced to the treatment site.

Beneficially, the misalignment allows for the guidewire 110 to access an internal channel 206 within the stent-graft as shown in FIG. 2C, and when the guidewire 110 is removed, the pressure of fluid (such as blood) from within the internal channel 206 directed outwardly causes the two layers, outer layer 202A and inner layer 202B, of the graft component 202 to be in contact with each other (e.g., the inner layer 202B is pushed against the outer layer 202A) to effectively form a temporary seal between the two layers 202A and 202B. The temporary seal can be re-opened at a later time by navigating the guidewire 110 through the two layers, outer layer 202A and inner layer 202B, and the two openings, outer aperture 204A and inner aperture 204B, as shown. The guidewire 110 may navigate in a substantially zigzag pattern to achieve the re-accessing of the internal channel 206 within the stent-graft.

In some examples, the implantable medical device 106 may be made using one or more elastomeric materials that may facilitate the ability to access (or re-access) the inner lumen of the medical device 106. Such access may be in association with a subsequent endoluminal procedure, where the clinician or other user desires to, again, delivery a guidewire and/or additional endoluminal devices through the same access site, and thus through the wall of the now-implanted medical device 106. To facilitate this ability to be re-accessed, while still functioning to subsequently help close off the access site, the implantable medical device 106 may be a braided device that is covered on the outside (e.g., partially or completely) with an elastomeric covering. In some examples, the seal is not a perfectly leakproof seal but facilitates a certain degree of tissue ingrowth into the surface material of the implantable medical device 106. For example, the seal in the implantable medical device 106 may reduce the flow of fluid entering the internal channel 206 of the implantable medical device 106 by at least 80%, by at least 85%, by at least 90%, by at least 95%, by at least 97%, by at least 99%, or any other suitable range therebetween. In some examples, the covering (e.g., graft material) may be a self-healing, or self-sealing material that substantially or completely seals following penetration by an endoluminal device (e.g., guidewire, introducer, catheter) and after all such devices have been removed from the penetration site.

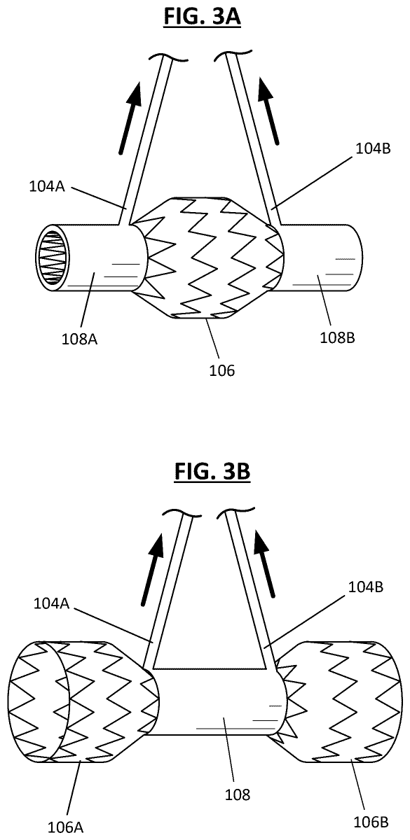

FIGS. 3A and 3B show two examples of how the implantable medical device 106 may be deployed and expanded using an implantable medical device system, according to embodiments disclosed herein. As referred to herein, the example of FIG. 3A is a middle-first expansion pattern, and the example of FIG. 3B is an edge-first expansion pattern.

In FIG. 3A (shown in a partially deployed state), the constraint 108 includes a first constraint 108A attached to a first deployment line 104A and a second constraint 108B attached to a second deployment line 104B. The implantable medical device 106 transitions from the constrained configuration to the expanded configuration by first expanding an intermediate portion 106C of the implantable medical device 106 located between a first end portion 106A and a second end portion 106B of the implantable medical device 106 when the first deployment line 104A and the second deployment line 104B are partially retracted, before expanding the first end portion 106A and the second end portion 106B of the implantable medical device 106 when the first deployment line 104A and the second deployment line 104B are further (or fully) retracted.

In FIG. 3B, the constraint 108 is a single constraint, and the first deployment line 104A and the second deployment line 104B are attached to two opposing ends of the single constraint 108. The implantable medical device 106 transitions from the constrained configuration to the expanded configuration by first expanding the first end portion 106A and the second end portion 106B of the implantable medical device 106 when the first deployment line 104A and the second deployment line 104B are partially retracted, before expanding an intermediate portion 106C of the implantable medical device 106 located between the first end portion 106A and the second end portion 106B when the first deployment line 104A and the second deployment line 104B are further (or fully) retracted. Although two deployment lines are shown, fewer (e.g., one) or greater (e.g., three or more) may be present and operate to actuate (release) the constraint. In examples including at least two deployment lines, the two or more deployment lines may be connected together such that actuating one of the lines may cause the other line(s) to be actuated simultaneously. As previously referenced, in various examples the constraint(s) are configured as one or more knit sleeves operable to unravel or deconstruct upon sufficient tensioning of the deployment line(s).

FIGS. 4A and 4B show two examples of how the implantable medical device 106 may be disposed with respect to the flexible hollow body 102, according to embodiments disclosed herein. The flexible hollow body 102 has a first end 102A (or proximal end) and a second end 102B (or distal end), and the implantable medical device 106 is pivotably disposed adjacent to the second end 102B of flexible hollow body 102. The deployment lines 104 are attached to a handle 400 disposed adjacent to the first end 102A of the flexible hollow body 102, and the handle 400 has a greater width than the flexible hollow body 102. In some examples, the handle 400 is in the form of a pull ring through which the clinician, or practitioner, may place a finger to pull back in order to retract the deployment lines 104. Beneficially, the width of the handle 400 being greater than the width of the flexible hollow body 102 or the width of the internal channel of the flexible hollow body 102 prevents the handle 400 from being inserted through the flexible hollow body 102, thereby providing a reliable grip of the practitioner.

In FIG. 4A, the longitudinal axes L1-L1 (of the flexible hollow body 102) and L2-L2 (of the implantable medical device 106) form an angle “A” at or near a distal end (or second end 102B) of the flexible hollow body 102 in an angled orientation relative to one another. The angle A may be flexibly changed or adjusted during deployment of the implantable medical device 106. The angle A may be adjustable from 0 degrees (FIG. 4B) to 180 degrees, 0 degrees to 90 degrees, 0 degrees to 45 degrees, or any angle or value therebetween or as otherwise desirable. For example, in FIG. 4B, the longitudinal axis L2-L2 of the implantable medical device 106 is aligned in a substantially parallel position with respect to the longitudinal axis L1-L1 of the flexible hollow body 102. The more angled position shown in FIG. 4A may be achieved, for example, by translating the implantable medical device 106 along the guidewire 110 (FIG. 1) which extends into the implantable medical device 106 at the midpoint 112 and protrudes from one end of the implantable medical device 106 as shown in FIG. 1. The implantable medical device 106 may track the guidewire 110 and pivot about or at the midpoint 112 of the implantable medical device 106, using the midpoint 112 as the pivot point, with respect to the flexible hollow body 102, or may simply flex and deform or otherwise transition to a more aligned position within the vessel as the device 106 tracks over the guidewire 110. While the guidewire 110 is present in various examples, in other examples the guidewire is not present and the medical device 106 simply tracks to the shape of the vessel in which it is being inserted (e.g., as it is introduced through an introducer sheath). In some examples, when the first deployment line 104A and the second deployment line 104B are both substantially taut, a position of the implantable medical device 106 relative to the flexible hollow body 102 is maintained. Moreover, tensioning of one or both of the deployment line(s) 104A, 104B may case the device to more readily transition from a more parallel, or 0 degree position with respect to flexible hollow body 102 to a more angled position relative to the flexible hollow body (e.g., to be aligned in a substantially perpendicular or orthogonal position with respect to a longitudinal axis (L1-L1) of the flexible hollow body 102 as shown in FIG. 1).

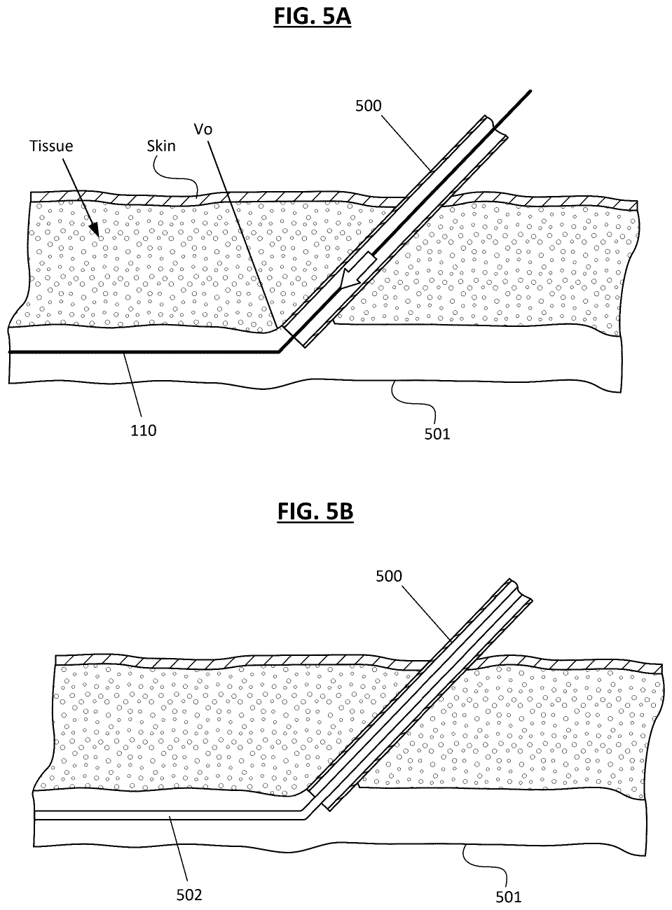

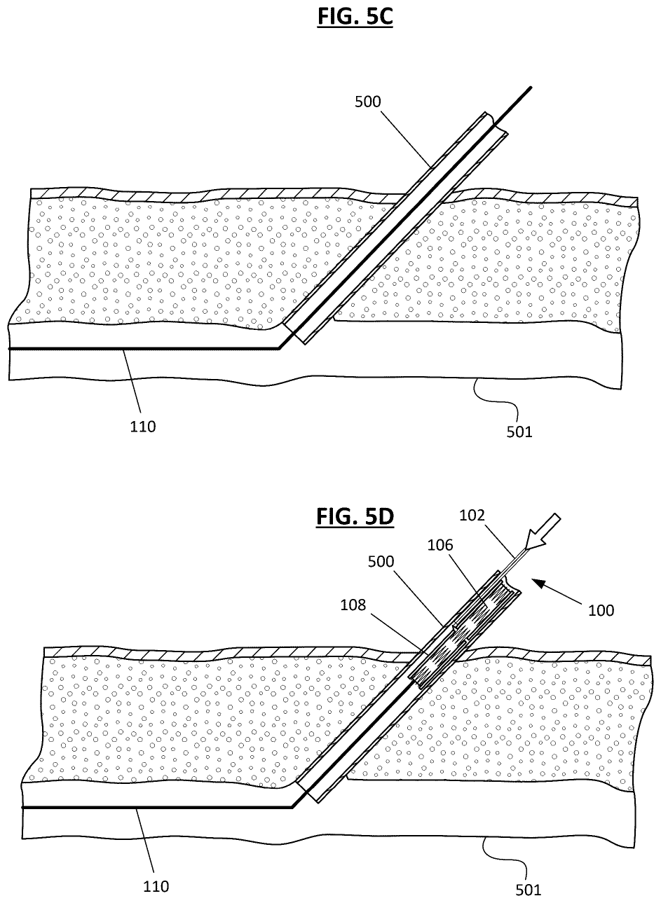

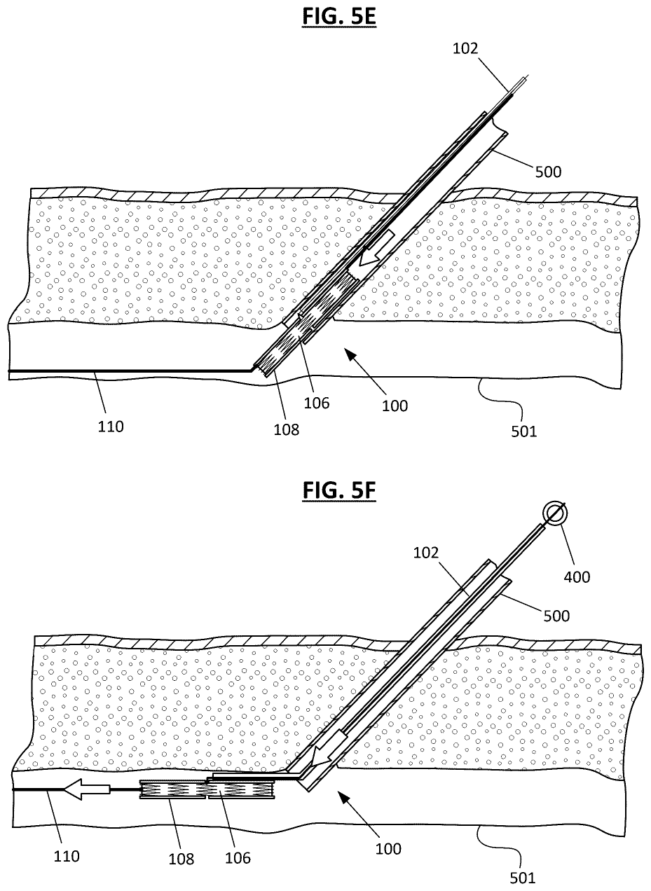

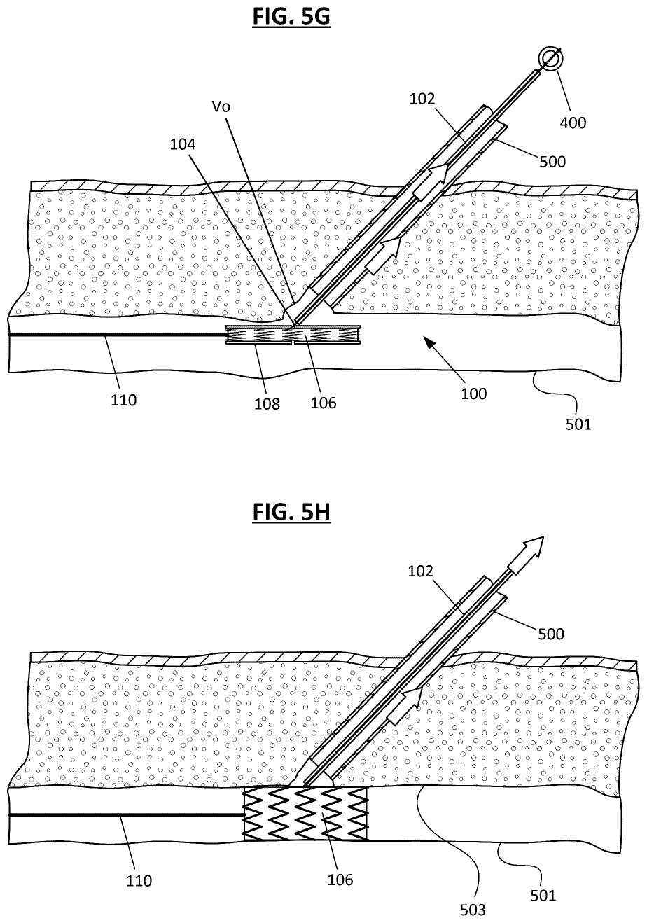

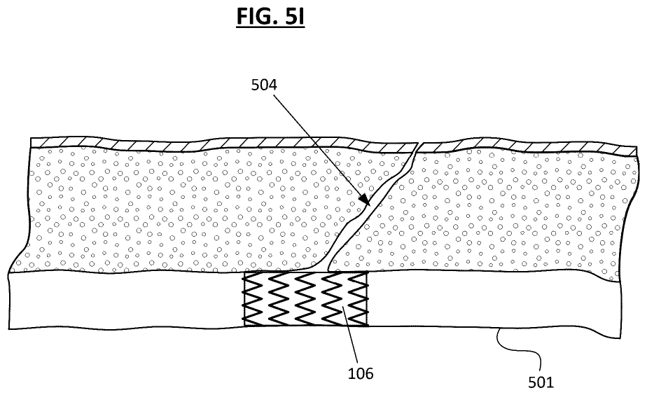

FIGS. 5A through 5I explain the steps of a surgical procedure or process in which the deployment device 100 may be implemented according to embodiments of an implantable medical device system disclosed herein. The surgical procedure of FIGS. 5A through 5I is an endoluminal, or catheter-based, procedure. The steps are shown for illustrative purposes only, and it is to be understood that some of the steps may be made optional or reordered with respect to the other steps in the process, as suitable. In the figures, the delivery methods and components are illustrated in a relatively schematic manner. For example, the guidewires and devices are show extending in straight, or a sharp angled fashion. Those of skill will appreciate that in practice, the devices will typically take on more curved, or rounded aspects, as opposed to sharp angles and straight, linear projections, in use. In general terms, the method includes forming a percutaneous access path into vasculature 501 (or vessel), with a path formed through skin and other tissue to an opening formed in a vessel wall Vo of the vasculature 501.

In FIG. 5A, a guidewire 110 is directed to pass into vasculature 501 including for example any suitable vessel (such as vein or artery) of the body, extending percutaneously into the vasculature 501. The guidewire 110 provides access for additional implantable medical devices to be transported along the guidewire and into the vessel (such as vein or artery), and as such, the guidewire 110 may be used to direct any suitable medical devices, including but not limited to stent-grafts, toward the target location with the vasculature. An introducer 300 may also be directed into the vasculature 501 along the guidewire 110, or vice versa, such that the guidewire 110 is disposed inside an internal channel of the introducer 300. Specifically, an introducer 300 is inserted into the body through the skin and the tissue underneath along the guidewire 302, as shown, until the introducer 300 reaches the vasculature 501 such as a vessel, which may be a blood vessel such as an artery or vein, thus forming an access path for the vasculature. The introducer 300 may be directed in a direction that opposes the fluid flow within the vessel, such as blood flow. The introducer 500 may be any suitable device such as a tube, catheter, or any elongated device with an internal channel having sufficient dimensions to fit inside the therapeutic device(s) required during the intervention and the deployment device 100. The introducer 500 is sized according to the size or shape of the tool, implant or other endoluminal device that is being introduced with in the vasculature. In some embodiments, the implant may include, but is not limited to, GORE® TAG® thoracic stent grafts (W. L. Gore & Associates, Inc.) or any other suitable stent grafts, for example. The vein or artery has a blood flow, and the introducer 500 may be directed or angled such that the end of the introducer 500 is directed in an opposite direction from the blood flow (retrograde) or in the same direction as blood flow (antegrade).

In FIG. 5B, a catheter 502 (shown generically and hereinafter referred to as an intervention catheter) may be inserted along the guidewire 110, or another guidewire, by positioning the catheter 502 around the guidewire 110 and distally moving the catheter 502 along the guidewire 110 until a distal tip (not shown) of the catheter 502 reaches a predetermined location. The catheter 502 may be used for transporting objects or fluids through the channel internal to the catheter 502. In some examples, the catheter 502 may be or include a large-bore closure device (such as any suitable suture device that is capable of closing arteriotomy sites between 8.5 and 10 F, for example) or an aortic device, among other suitable devices.

In FIG. 5C, the catheter 502 is removed while the guidewire 110 remains in the original position. While the guidewire 110 is shown as being employed for the various steps, it is also contemplated that a different guidewire may be introduced or used for different steps of the procedure.

In FIG. 5D, the deployment device 100 is coupled with or mounted along the guidewire 110 such that the guidewire 110 is disposed through the flexible hollow body 102 of the deployment device 100 and through a portion of the implantable medical device 106. The implantable medical device 106 is pivotably disposed adjacent to the flexible hollow body 102. The constraint 108 is disposed around and constraining the implantable medical device 106 in a constrained configuration. The one or more deployment lines 104 extend through the flexible hollow body 102 and are attached to the constraint 108. The deployment device 100 is then advanced distally into the introducer 500 as shown by the arrow.

In FIG. 5E, the deployment device 100 reaches the distal end of the introducer 500, at which point a portion of the implantable medical device 106 enters the vasculature 501 while in the constrained configuration, as the deployment device 100 is further pushed or inserted distally as shown by the arrow.

In FIG. 5F, the deployment device 100 adjusts in position (e.g., naturally or self-biased to arrange itself aligned to the vessel by its shape factor) according to the direction in which the guidewire 110 is extending. For example, as shown in the figure, the guidewire 110 curves or bends into the vasculature 501 when the guidewire 110 exits the distal end of the introducer 500. As such, the deployment device 100, or more specifically the flexible hollow body 102 and the implantable medical device 106 that is coupled with or attached to the flexible hollow body 102 using the deployment line(s) 104, is also redirected toward the same direction as the guidewire 110, as shown by the arrows. Until this point, the flexible hollow body 102 and the implantable medical device 106 are positioned substantially parallel to each other, or the implantable medical device 106 is aligned along the axis L1-L1 of the flexible hollow body 102.

In FIG. 5G, the deployment device 100 is left inside the vasculature 501 as the introducer 500 is retracted as shown by the arrow. The implantable medical device 106 is also partially retracted (for example by pulling on the handle 400) along the guidewire 110 as shown by the arrow. The partial retraction may include partially retracting the flexible hollow body 102 and/or the deployment line(s) 104. The partial retraction of the implantable medical device 106 along the guidewire 110 causes the implantable medical device 106 to center or align with respect to the opening in the vessel wall Vo. Depending on the vessel shape/tortuosity, the ability of the medical device 106 to pivot by any suitable degree or angle with respect to the flexible hollow body 102 will also help ensure proper alignment within the vessel during retraction. As such, partially retracting one or more of the flexible hollow body 102, the deployment lines 104, or the implantable medical device 106 along the guidewire 110 causes the pivoting of the implantable medical device 106 with respect to the flexible hollow body 102, for example at the midpoint 112 of the implantable medical device 106.

As explained above, in some examples, deploying the implantable medical device 106 may cause the implantable medical device 106 to transition from the constrained configuration to the expanded configuration by first expanding the intermediate portion 106C when the first deployment line 104A and the second deployment line 104B are partially retracted, before expanding the first end portion 106A and the second end portion 106B of the implantable medical device 106 when the first and second deployment lines 104A and 104B are further (or fully) retracted. In some examples, deploying the implantable medical device 106 may cause the implantable medical device 106 to transition from the constrained configuration to the expanded configuration by first expanding the first end portion 106A and the second end portion 106B when the first deployment line 104A and the second deployment line 104B are partially retracted, before expanding an intermediate portion 106C when the first and second deployment lines 104A and 104B are further (or fully) retracted.

In FIG. 5H, the implantable medical device 106 is deployed by further retracting the deployment lines 104 to cause the constraint 108 to fully release the implantable medical device 106 so as to transition from the constrained configuration to an expanded configuration with a greater cross-sectional area or width than the constrained configuration. For example, when the deployment lines 104 are further retracted, as shown by the arrow, the retraction causes the constraint 108 to be retrieved, thereby allowing the implantable medical device 106 to self-transition or self-expand to substantially fill the vasculature 501 as shown (or against a wall 503 of the vasculature 501), which may cause the device to center itself both radially and circumferentially. Subsequently, the flexible hollow body 102 may be retrieved, followed by the introducer 500, as shown by the arrow, resulting in only the guidewire 110 and the expanded implantable medical device 106 being left in the vasculature 501.

In FIG. 5I, the guidewire 110 is retrieved, leaving only the expanded implantable medical device 106 in the location of the vasculature 501 proximal to a tissue tract 504 formed by the introducer 500 being inserted through the skin and tissue underneath. The deploying of the implantable medical device 106 may cause substantial closure of the tissue tract 504 proximal to the vasculature. This resulting closure may be used as a replacement for, or as an additional method to use of other occluding mechanisms, such as collagen inserts or other occlusive material in the access site wound.

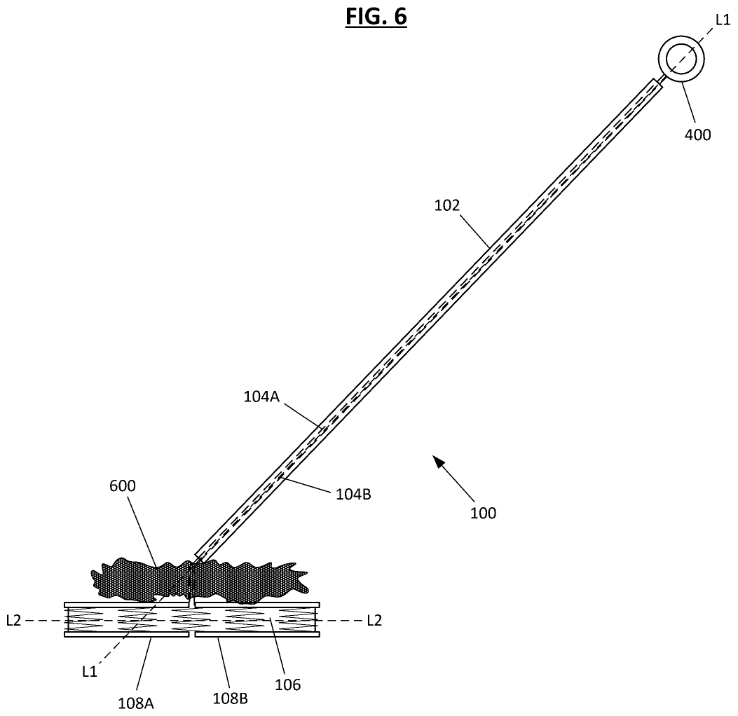

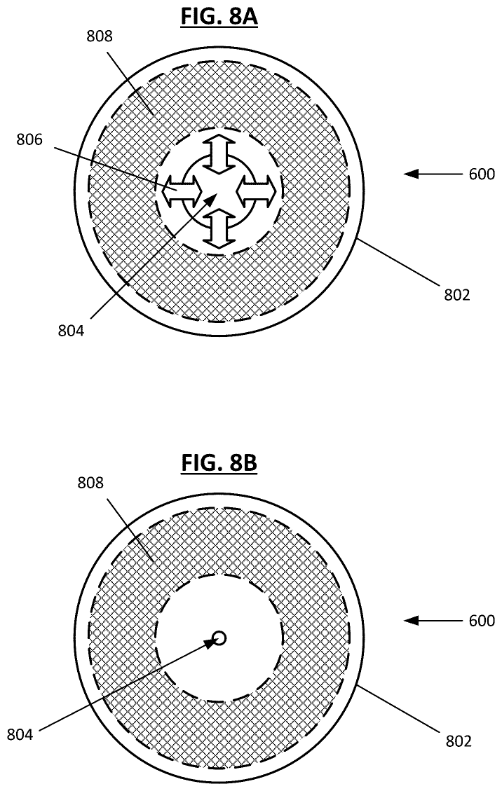

FIG. 6 shows an example of a deployment device 100 according to embodiments disclosed herein, where an implantable closure device 600 is disposed between the flexible hollow body 102 and the implantable medical device 106. As shown in FIGS. 8A and 8B, the implantable closure device 600 includes a body 802 made of a bioabsorbable material and having an opening 804 that is distensible in one or more directions, and the body 802 self-transitions from a first configuration (as shown in FIG. 8A) to a second configuration (as shown in FIG. 8B) in the absence of a force 806 being applied to open the opening 804.

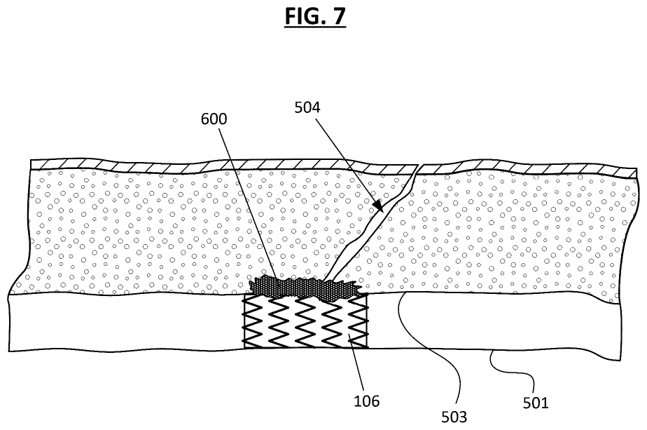

The opening 804 is substantially open in the first configuration and substantially closed in the second configuration to facilitate tissue ingrowth, while the implantable closure device 600 is disposed at the intersection of the vasculature 501 (vein or artery) and the tissue tract 504, as shown in FIG. 7.

In some examples, the body 802 of the implantable closure device 600 may be made of any suitable bioabsorbable material that facilitates or promotes tissue ingrowth after the device 600 is implanted. For example, the bioabsorbable material may include but is not limited to: copolymers and homopolymers of poly (α-hydroxy esters), such as copolymers of poly(lactic-co-glycolic acid) (PLGA), poly(glycolic acid) (PGA), and poly(lactic acid) (PLA); trimethylene carbonate (TMC); copolymers of PLA and TMC (PLA:TMC), copolymers of PGA and TMC (PGA:TMC) and copolymers of PLGA and TMC; and combinations thereof. In some examples, the material of the body 802 may be biocompatible, antibacterial, anti-inflammatory, and/or conductive to the body's healing process. Various other materials may be implemented that exhibit certain properties for facilitating at least some of the properties and functionalities of the bioabsorbable material after processing. At least some of the properties that facilitate the final functionality and properties of the bioabsorbable material are described herein in more detail.

In some examples, the implantable closure device 600 further includes a biocompatible, saline-activated or blood-activated adhesive 808 applied to a surface of the body 802, the adhesiveness of which is activated in response to being exposed to an environment that is filled with blood or saline solution. In such cases, the adhesive 808 is activated when the implantable closure device 600 is in a blood-filled environment. In some examples, the adhesive 808 may be dry (or solid); in some examples, the adhesive 808 may be wet (or liquid). The adhesive 808 may be applied between the body 802 of the implantable closure device 600 and a surrounding tissue or vessel wall 503 of the vasculature 501 (vein or artery) such that exposing the adhesive 808 to the blood within the vasculature is sufficient to activate the adhesive, causing the body 802 to be attached to the tissue wall surrounding the tissue tract 504.

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

What is claimed is:

1. An implantable medical device system comprising:
an implantable medical device having a midpoint along a length of the implantable medical device;
at least one constraint disposed around and constraining the implantable medical device in a constrained configuration;
a deployment device including a flexible hollow body, the implantable medical device positioned at an end of the flexible hollow body; and
one or more deployment lines attached to the at least one constraint, wherein at least one of the one or more deployment lines extend along the flexible hollow body and past a distal end of the flexible hollow body to operatively connect with the at least one constraint along the midpoint of the implantable medical device,
wherein the implantable medical device and the flexible hollow body are operable to transition from a substantially parallel orientation with the implantable medical device extending next to the flexible hollow body to an angled orientation relative to one another, and optionally a substantially perpendicular orientation, and
wherein retracting the one or more deployment lines is operable to cause the at least one constraint to release the implantable medical device to transition from the constrained configuration to an expanded configuration with a greater cross-sectional area than the constrained configuration.

2. The system of claim 1, wherein the at least one constraint includes a first constraint attached to a first deployment line of the one or more deployment lines and a second constraint attached to a second deployment line of the one or more deployment lines.

3. The system of claim 2, wherein the implantable medical device transitions from the constrained configuration to the expanded configuration by first expanding an intermediate portion of the implantable medical device located between a first end portion and a second end portion of the implantable medical device when the first deployment line and the second deployment line are partially retracted, before expanding the first end portion and the second end portion of the implantable medical device when the first deployment line and the second deployment line are fully retracted.

4. The system of claim 1, wherein the at least one constraint is a single constraint, and a first deployment line of the one or more deployment lines and a second deployment line of the one or more deployment lines are attached to two opposing ends of the single constraint.

5. The system of claim 4, wherein the implantable medical device transitions from the constrained configuration to the expanded configuration by first expanding a first end portion and a second end portion of the implantable medical device when the one or more deployment lines are partially retracted, before expanding an intermediate portion of the implantable medical device located between the first end portion and the second end portion when the one or more deployment lines are fully retracted.

6. The system of claim 1, wherein the implantable medical device has a first end portion and a second end portion, and the implantable medical device includes a first radiopaque marker disposed at or near the first end portion and a second radiopaque marker disposed at or near the second end portion.

7. The system of claim 1, wherein the implantable medical device comprises a self-expanding stent-graft.

8. The system of claim 7, wherein the stent-graft includes a graft component comprising an outer layer and an inner layer, the outer layer includes at least one outer aperture, the inner layer includes at least one inner aperture, and the outer aperture and the inner aperture are longitudinally misaligned with respect to each other.

9. The system of claim 8, further comprising a guidewire that is insertable into an internal channel of the implantable medical device through the outer layer and the inner layer.

10. The system of claim 9, wherein the flexible hollow body has a first end and a second end, and the implantable medical device is pivotably disposed adjacent to the second end of flexible hollow body.

11. The system of claim 10, wherein the one or more deployment lines are attached to a handle disposed adjacent to the first end of the flexible hollow body, wherein the handle has a greater width than the flexible hollow body.

12. The system of claim 11, wherein the handle is a pull ring.

13. The system of claim 1, further comprising an implantable closure device disposed between the flexible hollow body and the implantable medical device, the implantable closure device comprising a body made of a bioabsorbable material and having an opening that is distensible in one or more directions, wherein the body self-transitions from a first configuration to a second configuration in the absence of a force applied to open the opening, wherein the opening is substantially open in the first configuration and substantially closed in the second configuration.

14. The system of claim 13, wherein the implantable closure device further includes a biocompatible adhesive applied to a surface of the body.

15. The system of claim 9, further comprising a removable guidewire tube through which the guidewire is disposed, wherein the removable guidewire tube is withdrawn from the implantable medical device or the flexible hollow body prior to the implantable medical device or the flexible hollow body being advanced to a treatment site.

16. A method for delivering and deploying an implantable medical device system into vasculature of a patient to close an opening formed in a vessel wall of the vasculature, the method comprising:
extending a guidewire percutaneously into the vasculature through the opening formed in the vessel wall;
delivering an implantable medical device through the opening into the vasculature along the guidewire using a deployment device coupled to a midpoint of the implantable medical device, wherein the guidewire is received through a flexible hollow body of the deployment device and through a portion of the implantable medical device, the implantable medical device initially being substantially parallel to the flexible hollow body and then tracking over the guidewire to longitudinally align with the vasculature during delivery into the vasculature after the implantable medical device passes through the opening;
centering the implantable medical device on the opening formed in the vessel wall; and
deploying the implantable medical device from a constrained configuration to an expanded configuration to occlude the opening in the vessel wall and while permitting blood flow through a lumen of the implantable medical device.

17. The method of claim 16, wherein the deploying the implantable medical device causes substantial occlusion of a tissue tract proximal the opening in the vessel wall.

18. The method of claim 16, wherein the deployment device includes at least one constraint maintaining a first portion of the implantable medical device in the constrained configuration prior to deployment of the implantable medical device, the at least one constraint being attached to one or more deployment lines, and further wherein deploying the implantable medical device includes tensioning the one or more deployment lines.

19. The method of claim 18, wherein the deploying the implantable medical device causes the implantable medical device to transition from the constrained configuration to the expanded configuration by first expanding an intermediate portion of the implantable medical device located between a first end portion and a second end portion of the implantable medical device when the one or more deployment lines are partially retracted, before expanding the first end portion and the second end portion of the implantable medical device when the one or more deployment lines are fully retracted.

20. The method of claim 18, wherein the at least one constraint is a single constraint, and a first deployment line of the one or more deployment lines and a second deployment line of the one or more deployment lines are attached to two opposing ends of the single constraint.

21. The method of claim 20, wherein the deploying the implantable medical device causes the implantable medical device to transition from the constrained configuration to the expanded configuration by first expanding a first end portion and a second end portion of the implantable medical device when the first deployment line and the second deployment line are partially retracted, before expanding an intermediate portion of the implantable medical device located between the first end portion and the second end portion when the first deployment line and the second deployment line are fully retracted.

22. The method of claim 16, wherein the implantable medical device further includes a stent graft that is transitionable from the constrained configuration to the expanded configuration and the system further includes a body made of a bioabsorbable material having an opening that is distensible in one or more directions, the body being configured to self-transition from a first configuration to a second configuration during deployment of the implantable medical device, wherein the opening of the body is substantially open in the first configuration and substantially closed in the second configuration, and further wherein following deployment of the stent graft the body made of the bioabsorbable material is positioned in the tissue tract and/or between the stent graft and the vessel wall.
